(19) United States
(12) Patent Application Publication
(10) Pub. No.: US 2017/0202636 A1
(43) Pub. Date: Jul. 20, 2017

(54) INVESTIGATION AND CONTROL DEVICE OF DRIVE SYSTEM

(71) Applicants: Jerry T. Huang, Kaohsiung City (TW); EPED INC., Kaohsiung City (TW)

(72) Inventors: Ta-Ko Huang, Kaohsiung City (TW); Zong-Han Lyu, Kaohsiung City (TW); Jerry T. Huang, Kaohsiung City (TW)

(21) Appl. No.: 15/475,166

(22) Filed: Mar. 31, 2017

Related U.S. Application Data
(63) Continuation-in-part of application No. 14/828,064, filed on Aug. 17, 2015, now abandoned.

(30) Foreign Application Priority Data
Feb. 4, 2015 (TW) 104103710

(51) Int. Cl. A61C 1/00; A61B 34/20

(52) U.S. Cl. CPC A61C 1/0015; A61B 34/20; A61B 2034/2046

(57) ABSTRACT

An investigation and control device of a drive system is provided, which controls a power source of a surgical instrument by using a device for drive investigation and control, where the device for drive investigation and control receives a signal of the device for guide positioning and controls, according to a tracked connection state of the device for guide positioning and the surgical instrument, whether the surgical instrument is driven; and a control panel on the device for drive investigation and control electrically connects an device for instrumentation switch sensing and at least one practice model sensing device, so that the device for drive investigation and control controls, according to whether the instrumentation switch or/and the practice model is/are positioned correctly, whether the surgical instrument is normally driven, thereby completely and correctly controlling a correspondence between the surgical instrument and peripheral devices.

This is a continuation-in-part of co-pending U.S. patent application Ser. No. 14/828,064 filed on Aug. 17, 2015 and owned by the present applicant.

### CROSS-REFERENCE TO RELATED APPLICATION

This application claims the benefit of Taiwan Patent Application No. 104103710, filed on Feb. 4, 2015, which is hereby incorporated by reference for all purposes as if fully set forth herein.

### (a) TECHNICAL FIELD OF THE INVENTION

The present invention relates to an investigation and control device of a drive system, and particularly to an investigation and control device that can make guidance and operation records of a device for guide positioning consistent with an actual situation through investigation and control by means of drive control.

### (b) DESCRIPTION OF THE PRIOR ART

Current technologies for surgeries such as bone surgeries, abdominal surgeries and dental surgeries are interactive so that positions of medical devices are tracked. An aided-navigation dental treatment system uses a computer-aided navigation or tracing system to record a patient's teeth or jaw area by using a volume data set through, and positions of medical instruments and devices relative to the computer-aided navigation or tracing system are recorded.

In most dental guide positioning system technologies, a three-dimensional computed tomography (CT) image is used for photographing; a digital model of an area where a tooth is to be implanted is reconstructed; an optical tracking system is imported, and a position of a hand tool is tracked in an optical tracking manner; the image is processed in conjunction with use of the space positioning technology, so as to track an actual dental treatment area; coordinates of the two are connected; and the position of the hand tool is displayed in the CT image in real time, so that a depth and an angle of the hand tool is displayed in real time to a user during a dental training or dental surgery, and the user is provided with real-time data to determine whether the tool is out of alignment.

A dental teaching simulation machine or treatment table equipped with the action tracking and detection, positioning and guiding system can implement a three-dimensional digital record of a process of a surgery or practice operation. A surgical process including tooth turning, tooth implantation or injection requires accurate surgical operation behaviors, and the digital record can be used for guidance and indication during a surgery or operation recording and skill evaluation after a surgery. However, such a tracking and detection and positioning system has the following problems: due to limitations of the system, for example, the positioning system is an optical system, if there is a block between a tracking device and a hand tool, the whole system cannot perform detection and obtain a current related operation record, so that the operation process cannot be 100% consistent with the record. Therefore, guidance, operation recording and evaluation for a surgery or practice cannot be totally consistent with an actual situation.

### SUMMARY OF THE INVENTION

An objective of the present invention is to provide an investigation and control device of a drive system, where a surgical instrument can be normally driven only when a guide positioning system, an device for instrumentation switch sensing or/and a practice model sensing device are put in right actuation positions through the investigation and control device.

To solve the problems in the prior art, the present invention relates to an investigation and control device of a drive system, used in a machine for dental treatment with a device for guide positioning, where the machine for dental treatment is provided with a surgical instrument, used for clinical or practice operations, and the surgical instrument is provided with a element for positioning corresponding to the device for guide positioning, characterized by including: a device for drive investigation and control electrically connected between the machine for dental treatment and the surgical instrument, where the device for drive investigation and control receives a signal of the device for guide positioning and controls, according to a tracked connection state of the device for guide positioning and the surgical instrument, whether the surgical instrument is normally driven; and a control panel electrically connected to the device for drive investigation and control, where the control panel electrically connects an device for instrumentation switch sensing and at least one practice model sensing device, which are used for controlling an instrumentation switch and a practice model through investigation, so that the device for drive investigation and control controls, according to whether the instrumentation switch or/and the practice model is/are positioned correctly, whether the surgical instrument can be normally driven.

The device for drive investigation and control may be further disposed inside the machine for dental treatment, and the device for drive investigation and control is electrically connected with the device for guide positioning and controls, according to the tracked connection state of the device for guide positioning and the surgical instrument, whether the surgical instrument is normally driven by the machine for dental treatment.

The device for guide positioning has action tracking and detection, positioning and guiding functions, and when the device for guide positioning can track and detect the surgical instrument, the device for drive investigation and control enables the machine for dental treatment to normally drive the surgical instrument; and when the device for guide positioning cannot track the surgical instrument and detects that the surgical instrument is abnormally disconnected, the device for drive investigation and control cuts off a power source through which the machine for dental treatment drives the surgical instrument.

When the power source through which the surgical instrument is driven is fluid-driven, the device for drive investigation and control controls circulation and blocking of a fluid flowing into a pipeline of the surgical instrument.

When the power source through which the surgical instrument is driven is electric-driven, the device for drive investigation and control controls connection and disconnection of the power source to the surgical instrument.

The surgical instrument can be driven only when the control panel detects that the instrumentation switch equipped with the device for instrumentation switch sensing is correctly turned on and positioned.

The surgical instrument can be normally driven only when the control panel detects that the practice model sensing device is correctly installed and positioned.

The present application has the following advantages: the device for drive investigation and control receives a signal of the device for guide positioning, so that the surgical instrument can be normally driven only when the guide positioning system cannot perform tracking and positioning due to a block or signal interference between the tracking device and the surgical instrument, when the device for guide positioning cannot track the surgical instrument and detects that the surgical instrument is abnormally disconnected, and when the control panel detects that the instrumentation switch is correctly turned on and positioned, or after it is detected in practice that the practice model is correctly installed and positioned. Some instruments of the system are stopped by temporarily cutting off the driving power source of the surgical instrument through investigation and control, and the driving power source of the surgical instrument is restored after the device for guide positioning is capable of tracking and detection and the peripheral devices have been disposed at proper positions, thereby completely and correctly controlling a correspondence between the surgical instrument and the peripheral devices. The surgical instrument can be normally driven only at right moment, so that digital recorded data is made consistent with an actual operation through investigation and control.

### BRIEF DESCRIPTION OF THE DRAWINGS

FIG. 1 is a schematic system view of an embodiment of the present application; and

FIG. 2 is a schematic system view of another embodiment of the present application.

### DETAILED DESCRIPTION OF THE PREFERRED EMBODIMENTS

Detailed contents and technical description about the present invention are further described below with embodiments, but it should be understood that the embodiments are merely for illustration and should not be construed as limitations on implementation of the present invention.

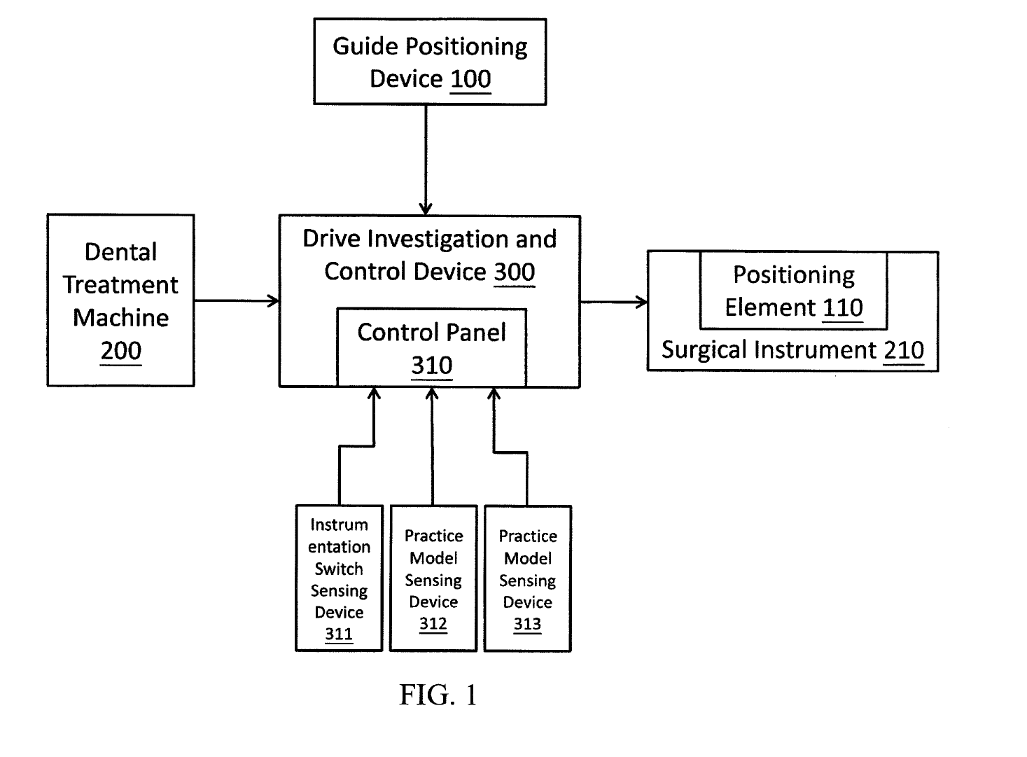

Referring to FIG. 1, FIG. 1 is a schematic system view of an embodiment of the present application. The present application relates to an investigation and control device of a drive system, used in a machine 200 for dental treatment with a device 100 for guide positioning. The device 100 for guide positioning has action tracking and detection, positioning and guiding functions, and can implement a three-dimensional digital record of a process of a surgery or practice operation. The machine 200 for dental treatment is provided with a surgical instrument 210, used for clinical or practice operations, and the surgical instrument 210 is provided with an element 110 for positioning corresponding to the device 100 for guide positioning. Tracking and positioning are performed in an optical manner, an image is processed in conjunction with the space positioning technology, and a patient's oral and/or practice head model is positioned by using a tracking tool, so as to provide guidance and indication during a surgery or operation recording and skill evaluation after a surgery so that a mistake during usage can be corrected later. The device 100 for guide positioning is a known prior art in the medical guide positioning system, which is not repeated herein. It is noted here that the element 110 for positioning mentioned above is an element for light reflection or element for active light emission, where the device 100 for guide positioning can calculate a position of the surgical instrument 210 in a space according to the position of the element 110 for positioning to obtain the positioning mentioned above by tracking the spot generated by the element for light reflection or element for active light emission and according to the position relationship of the spot corresponding to the surgical instrument 210 stored in the device 100 for guide positioning.

Provided is a machine 200 for dental treatment and a surgical instrument 210, extending from the machine 200 for dental treatment, to be used in a patient's oral and/or practice head model (not shown in the figure) for oral surgery or practice operations, for example, a dental handpiece, an electric-driven syringe, a tooth implantation machine and a tooth cleaning machine. The surgical instrument 210 held in hand by a physician or an intern is provided with the element 110 for positioning corresponding to the device 100 for guide positioning, so that the device 100 for guide positioning can track, detect and position actions of the surgical instrument 210. The machine 200 for dental treatment is a machine allowing a patient to lie down on it for dental treatment, which is configured with a liftable recliner and coupled to the surgical instrument 210 for a dentist to perform a corresponding treatment to a patient.

A device 300 for drive investigation and control is electrically connected between the machine 200 for dental treatment and the surgical instrument 210, where the device 300 for drive investigation and control receives a signal sent from the device 100 for guide positioning; in implementation applications, the signal may be received by using a wire or an existing known wireless technology, such as Bluetooth and wifi. The device 300 for drive investigation and control is in signal connection with the machine 200 for dental treatment and surgical instrument 210, and the machine 200 for dental treatment and surgical instrument 210 are controlled to start or stop correspondingly through a signal, where whether they are started or stopped is determined by a state detected by a sensor in connection therewith.

The device 300 for drive investigation and control controls, according to a tracked connection state of the device 100 for guide positioning and the surgical instrument 210, whether the surgical instrument 210 is normally driven. When the device 100 for guide positioning can track and detect the action of the surgical instrument 210 and the device 100 for guide positioning and the surgical instrument 210 are normally connected, that is, no block or signal interference occurs between the tracking device of the device 100 for guide positioning and the surgical instrument 210 to affect tracking and positioning, the device 300 for drive investigation and control enables the machine for dental treatment 200 to normally drive the surgical instrument 210. When the device 100 for guide positioning cannot track and detect the surgical instrument 210 and the device 100 for guide positioning and the surgical instrument 210 are abnormally disconnected, that is, a block occurs between the tracking device of the device 100 for guide positioning and the surgical instrument 210, the block may be in the device or it may be that a user (physician or intern) blocks a connecting path, the device 300 for drive investigation and control cuts off a power source through which the surgical instrument 210 is driven.

Also, a control panel 310 is electrically connected to the device 300 for drive investigation and control, where the control panel 310 is used for electrically connecting a device 311 for instrumentation switch sensing and at least one practice model sensing device 312, 313. The practice model sensing devices 312, 313 may be sensing devices of dental practice peripheral models such as maxillary and mandibular models for practice, dental models for tooth implantation practice, prosthesis practice single tooth, and injection site models. In implementation, the device for drive investigation and control 300 controls, according to whether the device 311 for instrumentation switch sensing or/and the practice model sensing devices 312, 313 (in FIG. 1, two sensing devices are taken as an example; in implementation, the number of sensing devices connected to a port of the control panel 310 may be increased or decreased according to a requirement on the number of connected sensing devices) is/are correctly positioned, whether the surgical instrument 210 is normally driven. The device 311 for instrumentation switch sensing is a conventional device allowing a physician to treadle a pedal with foot to drive related equipment.

The device 311 for instrumentation switch sensing is disposed at an instrumentation switch, such as a pedal switch or a shift switch used in the dental handpiece, while an electric pressing switch used in a syringe is controlled and started by a user (physician or intern), and the surgical instrument 210 can be driven after the user (physician or intern) completes preparation and correctly starts the instrumentation switch, which prevents unnecessary waste of power sources and prevents non-user-induced incorrect driving.

When a practice head model is used, the control panel 310 detects whether the maxillary and mandibular sensing devices 312, 313 are correctly installed, for example, only when a maxillary sensing device and a mandibular sensing device are correctly installed and positioned in the practice head model, the surgical instrument 210 can be normally driven. In implementation, the sensing devices may be reed sensing switches, Hall effectors, press (short circuit) switches, light interrupt switches or other sensing switches that have been technically mature in the market and have installation sensing functions. For example, a reed sensing switch is added to the maxillary sensing device and the mandibular sensing device, to detect, through magnetic attraction and sensing of the head model, whether the maxillary sensing device and the mandibular sensing device are separated from the practice head model, and the practice environment of the intern, and whether the devices in the practice head model are correctly arranged, so that the intern can practice in a correct practice situation. For example, turning can be performed only when the dental model is placed in the head model, and teeth cannot be turned without taking out the dental model.

When the power source through which the surgical instrument 210 is driven is fluid-driven, the device 300 for drive investigation and control controls circulation and blocking of a fluid flowing into a pipeline of the surgical instrument 210. For example, the dental handpiece uses a pressure gas as a turning torque source, and in implementation, the device 300 for drive investigation and control can block a pipeline between the machine 200 for dental treatment and the surgical instrument 210 in a known mechanical or electromagnetic technical manner, to temporarily stop supplying a fluid that drives the surgical instrument 210.

When the power source through which the surgical instrument 210 is driven is electric-driven, the device 300 for drive investigation and control controls connection and disconnection of the power source to the surgical instrument 210. For example, the surgical instrument 210 is a motor, an electric-driven syringe, or another electronic-controlled surgical instrument, and in implementation, the device 300 for drive investigation and control can completely or partially disconnect the power supply between the machine 200 for dental treatment and the surgical instrument 210 in an electronic control manner, to temporarily stop supplying power that drives the surgical instrument 210.

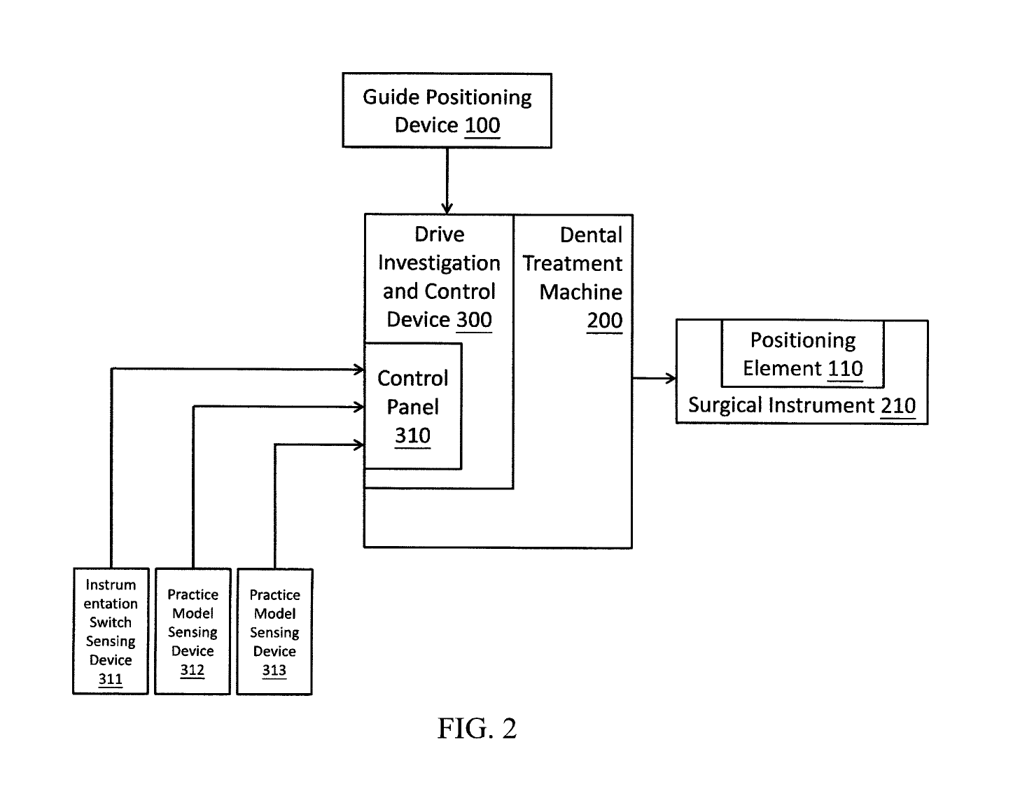

Referring to FIG. 2, in implementation of the present invention, the device 300 for drive investigation and control may also be directly disposed in the machine 200 for dental treatment, and control, according to a tracked connection state of the device 100 for guide positioning and the surgical instrument 210, whether the machine 200 for dental treatment provides a driving power source to the surgical instrument 210. When the device 100 for guide positioning can track the surgical instrument 210 and detects that the surgical instrument 210 is normally connected, the machine 200 for dental treatment drives the surgical instrument according to a normal operation state. However, when the device 100 for guide positioning cannot track the surgical instrument and detects that the surgical instrument is abnormally disconnected 210, the device 300 for drive investigation and control cuts off the power source of the machine 200 for dental treatment to the surgical instrument 210, so that the machine 200 for dental treatment loses part of the power source and cannot normally drive the surgical instrument 210.

In the same way, the instrumentation switch of the device 311 for instrumentation switch sensing is controlled and started by a user (physician or intern), and the machine 200 for dental treatment can normally drive the surgical instrument 210 only after the user (physician or intern) completes preparation and correctly starts the instrumentation switch.

As stated above, only when a practice head model is used and it is detected through the control panel 310 that the maxillary and mandibular sensing devices 312, 313, the machine for dental treatment 200 can normally drive the surgical instrument 210. The practice head mold allows learners to be familiar with head models used by the instrument before formal treatment of patents so that maxillary and mandibular sensing device 312, 313 may be installed directly, thereby determining whether the corresponding steps are completed, or whether the instrument reaches a corresponding position.

When the power source through which the surgical instrument 210 is driven is fluid-driven, the device 300 for drive investigation and control controls circulation and blocking of a fluid in the machine 200 for dental treatment. For example, the dental handpiece uses a pressure gas as a turning torque source, and in implementation, the device 300 for drive investigation and control can block a pipeline through which gas flows into the whole machine 200 for dental treatment in a known mechanical or electromagnetic technical manner, so that the machine 200 for dental treatment cannot supply a fluid that drives the surgical instrument 210.

When the power source through which the surgical instrument 210 is driven is electric-driven, the device 300 for drive investigation and control controls connection and disconnection of the power source to the machine 200 for dental treatment. For example, a motor, an electric-driven syringe or another electronic-controlled surgical instrument may completely disconnect the power supply of the machine 200 for dental treatment in an electronic control manner, so that the machine 200 for dental treatment cannot supply power that drives the surgical instrument 210.

It is worth mentioning that the selection between complete and partial disconnection of the power supply in implementation should be determined according to the state of the surgical instrument 210. Using a tooth implantation machine as an example, if the power supply is completely disconnected and then connected, the entire system cannot be quickly restarted for use; therefore, in implementation, it is feasible to install the device 300 for drive investigation and control on a motor line of the surgical instrument 210 for partial disconnection.

However, the above descriptions are merely preferred embodiments of the present invention, and cannot be used to limit the scope of implementation of the present invention. Any simple and equivalent variations and modifications made according to CLAIMS and SUMMARY of the present invention should fall within the scope of the present invention.

I claim:

1. An investigation and control device of a drive system, used in a machine for dental treatment with a device for guide positioning, wherein the machine for dental treatment is having a surgical instrument, used for clinical or practice operations, and the surgical instrument is provided with a element for positioning corresponding to the device for guide positioning, characterized by comprising:
a device for drive investigation and control electrically connected between the machine for dental treatment and the surgical instrument, wherein the device for drive investigation and control receives a signal of the device for guide positioning and controls, according to whether the device for guide positioning and the surgical instrument are in a trackable connection state, whether the surgical instrument can be driven; and
a control panel electrically connected to the device for drive investigation and control, wherein the control panel electrically connects an device for instrumentation switch sensing and at least one practice model sensing device, which are used for controlling an instrumentation switch and a practice model through investigation, so that the device for drive investigation and control controls, according to whether the instrumentation switch or/and the practice model is/are positioned correctly, whether the surgical instrument can be normally driven.

2. An investigation and control device of a drive system, used in a machine for dental treatment with a device for guide positioning, wherein the machine for dental treatment is having a surgical instrument, used for clinical or practice operations, and the surgical instrument is provided with a element for positioning corresponding to the device for guide positioning, characterized by comprising:
a device for drive investigation and control electrically connected to the inside of the machine for dental treatment, wherein the device for drive investigation and control receives a signal of the device for guide positioning and controls, according to whether the device for guide positioning and the surgical instrument are in a trackable connection state, whether the surgical instrument can be driven by the machine for dental treatment; and
a control panel electrically connected to the device for drive investigation and control, wherein the control panel electrically connects an device for instrumentation switch sensing and at least one practice model sensing device, which are used for controlling an instrumentation switch and a practice model through investigation, so that the device for drive investigation and control controls, according to whether the instrumentation switch or/and the practice model is/are positioned correctly, whether the surgical instrument can be normally driven by the machine for dental treatment.

3. The investigation and control device of a drive system according to claim 1, wherein the device for guide positioning has action tracking and detection, positioning and guiding functions, and when the device for guide positioning can track and detect the surgical instrument, the device for drive investigation and control enables the machine for dental treatment to normally drive the surgical instrument; and when the device for guide positioning cannot track the surgical instrument and detects that the surgical instrument is abnormally disconnected, the device for drive investigation and control cuts off a power source through which the machine for dental treatment drives the surgical instrument.

4. The investigation and control device of a drive system according to claim 2, wherein the device for guide positioning has action tracking and detection, positioning and guiding functions, and when the device for guide positioning can track and detect the surgical instrument, the device for drive investigation and control enables the machine for dental treatment to normally drive the surgical instrument; and when the device for guide positioning cannot track the surgical instrument and detects that the surgical instrument is abnormally disconnected, the device for drive investigation and control cuts off a power source through which the machine for dental treatment drives the surgical instrument.

5. The investigation and control device of a drive system according to claim 3, wherein, when the power source through which the surgical instrument is driven is fluid-driven, the device for drive investigation and control controls circulation and blocking of a fluid flowing into a pipeline of the surgical instrument.

6. The investigation and control device of a drive system according to claim 3, wherein, when the power source through which the surgical instrument is driven is electric-driven, the device for drive investigation and control controls connection and disconnection of the power supply to the surgical instrument.

7. The investigation and control device of a drive system according to claim 3, wherein the surgical instrument can be driven only when the control panel detects that the instrumentation switch equipped with the device for instrumentation switch sensing is turned on and positioned.

8. The investigation and control device of a drive system according to claim 3, wherein the surgical instrument can be normally driven only when the control panel detects that the practice model equipped with the practice model sensing device is installed and positioned.
